(19) United States
(12) Patent Application Publication
(10) Pub. No.: US 2023/0084046 A1
(43) Pub. Date: Mar. 16, 2023

(54) CONSUMER PRODUCT COMPRISING A PLURALITY OF MICROCAPSULES DEFINED BY THE MEAN TOTAL SURFACE AREA

(71) Applicant: GIVAUDAN SA, VERNIER (CH)

(72) Inventor: Emmanuel AUSSANT, Paris (FR)

(21) Appl. No.: 17/929,021

(22) PCT Filed: Mar. 15, 2021
(86) PCT No.: PCT/EP2021/056488

§ 371 Date: Aug. 10, 2022

(30) Foreign Application Priority Data
Mar. 18, 2020 (GB) 2003883.2

(51) Int. Cl. C11D 3/50; A61K 8/11; C11D 11/00; C11D 17/00; B01J 13/14

(52) U.S. Cl. CPC C11D 3/505; A61K 8/11; C11D 11/0017; C11D 17/0013; B01J 13/14; A61K 2800/412; A61K 2800/624

(57) ABSTRACT

The present invention relates to a consumer product comprising a plurality of microcapsules, to a method for obtaining a consumer product and to a use of a plurality of microcapsules in a such a consumer product.

The present invention relates to a consumer product comprising a plurality of microcapsules, to a method for obtaining a consumer product and to a use of a plurality of microcapsules in a such a consumer product.

It is known to incorporate encapsulated functional materials in consumer products, such as household care, personal care and fabric care products. Functional materials include for example fragrances, flavors, cosmetic ingredients, biocides, substrate enhancers and nutraceuticals.

Microcapsules that are particularly suitable for delivery of such functional materials are core-shell microcapsules, wherein the core comprises the functional material and the shell is impervious or partially impervious to the functional material. Usually, these microcapsules are used in aqueous media and the encapsulated functional materials are hydrophobic. A broad selection of shell materials can be used, provided these shell materials are impervious or partially impervious to the encapsulated functional material.

Among the functional materials, fragrances are encapsulated for a variety of reasons. Microcapsules can isolate and protect the fragrances from external suspending media, such as consumer product bases, with which they may be incompatible or unstable in. They are also used to assist in the deposition of fragrance ingredients onto substrates, such as skin, hair, fabrics or hard household surfaces. Furthermore, they can act as a means of controlling the spatio-temporal release of the fragrance. These features provide enhanced olfactive benefits that are currently not achievable without microcapsules.

Thermosetting resins are common shell materials for such fragrance encapsulates. Core-shell microcapsules formed from aminoplast resins, polyurea resins, polyurethane resins, polyacrylate resins and combinations thereof are generally quite resistant to fragrance leakage when dispersed in aqueous suspending media, even in surfactant-containing media. The average diameter of these microcapsules is typically from 1 to 100 μm, more particularly from 5 to 50 μm.

Microcapsules having such an average diameter may have the disadvantage of disturbing the optical aspect of consumer products, such as unstructured liquid detergents, shampoos and shower gels. Such optical disturbances include the occurrence of turbidity or visible dots in the product. This is especially problematic if the consumer product is supposed to be transparent or translucent.

WO 2016/170531 A1 describes nano-sized microcapsules, having a diameter smaller than 1 μm, which enable the formation of transparent suspensions. However, the industrial production of nano-sized capsules is known to be difficult, especially because of the high viscosity of slurries containing such microcapsules at industrially relevant solid contents. Furthermore, microcapsules below 1 μm size may have too large of a surface to volume ratio to retain the functional material comprised in the core or the microcapsule over time, especially if this functional material is soluble or volatile in the product base.

It is therefore a problem underlying the present invention to overcome the above-mentioned shortcomings in the prior art. In particular, it is a problem underlying the present invention to provide translucent or transparent consumer products comprising microcapsules, which do not have the above-mentioned disturbances their appearance, while still providing the desired olfactive benefits of the microcapsules.

By “transparent consumer product” it is meant a product having a turbidity of 50 NTU or less, preferably 20 NTU or less, more preferably 15 NTU or less, still more preferably 10 NTU or less. The abbreviation NTU stands for Nephelometric Turbidity Unit, a unit of turbidity well-known to and widely used by the art and measured with a suitable instrument.

By “translucent consumer product” is meant a product having a turbidity between 50 and 350 NTU, preferably between 50 and 300 NTU, more preferably between 50 and 200 NTU, still more preferably between 50 and 100 NTU.

In a first aspect, the present invention solves the above problem by providing a consumer product comprising a plurality of microcapsules. The microcapsules comprise a core and a shell around the core. The core comprises at least one functional material. The mean total surface area of the plurality of microcapsules comprised in one litre of consumer product is from 0.02 to 0.27 m², preferably from 0.02 to 0.12 m².

The applicant has surprisingly found that consumer products comprising microcapsules having a mean total surface area per litre of consumer product in the above-mentioned range have a turbidity value that is lower than 350 NTU, i.e. are transparent or translucent, whatever the corresponding mean diameter or the corresponding median volume number of microcapsules is. The mean total surface area of the plurality of microcapsules comprised in one litre of consumer product is the use an important parameter for obtaining the optimal level of functional material in the consumer product, while still keeping the consumer product transparent to translucent.

In preferred embodiments of the present invention, the mean total surface area of the plurality of microcapsules is calculated based on the surface mean diameter D(3,2) and on the volume median diameter Dᵥ(50) of the microcapsules, in particular by performing the steps of:
a) Measuring the surface mean diameter D(3,2) (also referred to as surface-volume mean diameter or Sauter diameter) and volume median diameter Dᵥ(50) of the microcapsules by static light scattering and obtaining therefrom the surface and surface mean radius R(3,2) and volume median radius Rᵥ(50) of the microcapsules;
b) Calculating the mean surface area of the microcapsules s_{c} based on the surface mean radius obtained in step a).

s_{c}=4π(3,2))² (Equation 1)
c) Calculating the median volume v_{c} of the microcapsules based on the volume median radius obtained in step a).

$${v}_{c} = \frac{4}{3} {π ( {R}_{v} ( 50 ) )}^{3} \qquad ( Equation 2 )$$

d) Calculating the volume median number of microcapsules included in this plurality of microcapsules.

Either by sampling the particle size distribution of the microcapsules present in the consumer product, calculating the volume distribution of the microcapsules and extracting the volume median number of microcapsules from this distribution;

Or by dividing the total volume of the core and shell materials of the microcapsules present in the consumer product (V_{core+shell}) by the median volume v_{c} of the microcapsule obtained in step c).

$${N}_{v} = \frac{{V}_{core + shell}}{{v}_{c}} \qquad ( Equation 3 )$$

The second approach is preferred in the context of the present invention.
e) Calculating the mean total surface area S of the plurality of microcapsules by multiplying the mean surface area of the microcapsules obtained in step b) with the volume median number of microcapsules obtained in step d).

S=Nᵥs_{c}=4πNᵥ(R(3,2))² (Equation 4)
Or respectively:

$$S = {N}_{v} {s}_{c} = ( \frac{3 ( {V}_{core + shell} )}{4 {π ( {R}_{v} ( 50 ) )}^{3}} ) 4 {π ( R ( 3 , 2 ) )}^{2} = \frac{3 ( {R ( 3 , 2 )}^{2} ) ( {V}_{core + shell} )}{{( {R}_{v} ( 50 ) )}^{3}} \qquad ( Equation 5 )$$

With respect to the second option of step d), the total volume of the core present in the consumer product may, formally, be calculated by dividing the nominal weight of core material m_{core} added to the consumer product in the form of encapsulated core by the density of this core material ρ_{core}. The total nominal volume of the shell may be calculated by dividing the nominal weight of shell material mₛₕₑₗₗ added to the consumer product in the form of encapsulating shell by the density ρₛₕₑₗₗ of this shell. The density of the core material may be measured experimentally at 25° C. by using any suitable method known to the art, whereas for the density of the shell material, an estimated density of 1.15±0.1 g/cm³ at 25° C. may be taken. This covers the density range of the encapsulating materials disclosed hereinafter. An uncertainty of ±10% is acceptable in the context of the present invention, because the microcapsule shell to core weight ratio is usually less than 0.2. Under these conditions, the impact of such an uncertainty on the calculated total surface area is less than 2%.

However, this calculation may be advantageously simplified by considering the fact that the microcapsules, in order to match the density of the consumer product base, have usually a known density of ρ_{caps}, for example 1.05±0.05 g/cm³ at 25° C. This value may be used to calculate the nominal volume of the microcapsule in Equation 1, in order to get Equation 1b:

$$S = \frac{3 {( R ( 3 , 2 ) )}^{2} {m}_{caps}}{{( {R}_{v} ( 50 ) )}^{3} {ρ}_{caps}} \qquad ( Equation 6 )$$

Emulsifiers, protective colloids and stabilizing polymers that are commonly used in the synthesis of the microcapsules or post-added to microcapsule slurries in order to prevent microcapsule aggregation, creaming or sedimentation are not taken into account in the above shell density.

The static light scattering method used for measuring both surface mean diameter D(3,2) and volume median diameter Dᵥ(50) of the microcapsules in step a) involves laser diffraction particle size analysis and the Mie scattering theory. The principle of the Mie theory and how static light scattering can be used to measure the mean and median diameters of a plurality of particles having a distribution of sizes can be found, for example in H. C. van de Hulst, Light scattering by small particles. Dover, N.Y., 1981. Both surface mean diameter D(3,2) and volume median diameter Dᵥ(50) may be calculated by using the software provided with the light scattering measurement apparatus.

In preferred embodiments of the present invention the volume median diameter Dᵥ(50) is from 5 to 50 μm, preferably from 8 to 35 μm, still more preferably from 10 to 20 μm. Microcapsules having a volume median diameter larger than 50 μm are not desired, because of the fact that individual microcapsules of that size may be visible for the human eye, while microcapsules below 5 μm may have a too large of a surface to volume ratio of to retain the functional material comprised in the core or the microcapsules over time, especially if this functional material is soluble in the consumer product or is volatile.

In particular embodiments of the present invention the consumer product has a turbidity lower than 350 NTU, preferably lower than 300 NTU, more preferably lower than 200 NTU, still more preferably lower than 100 NTU, even more preferably lower than 50 NTU at 25° C.

In context of the present invention, the turbidity may be measured by using a turbidimeter operating in nephelometric mode, wherein the intensity of the scattered light is measured at an angle of 90° and this value is divided by the intensity of the transmitted light, which is typically measured at an angle of from 160 to 180, depending on the instrument geometry. The incident light may be a white light, for example the light emitted by a so-called “white LED”, having, for example, a wavelength range of 400 to 680 nm. Alternatively, the incident light may be a monochromatic light having a specified wavelength in both the visible domain of the electromagnetic spectrum, e.g. 460 nm or 650 nm, or in the near infrared domain of the electromagnetic spectrum, e.g. 860 nm. Samples having a strong colouration are preferably measured with an incident light having near infrared wavelengths.

In particular embodiments of the present invention, the at least one functional material is selected from the group consisting of a fragrance, a flavour and a cosmetic ingredient, preferably a fragrance.

By “fragrance” is meant here a mixture of fragrance ingredients. By “fragrance ingredient” is meant chemical substance (or group of chemical substances) having an odour that may be employed in fragrances for the primary purpose to provide a contribution of a pleasant odour, either alone or in combination with other fragrance ingredients.

In preferred embodiments of the present invention, the core composition comprises at least one fragrance ingredient. A comprehensive list of fragrance ingredients that may be encapsulated in accordance with the present invention may be found in the perfumery literature, for example “Perfume & Flavor Chemicals” by S. Arctander (Allured Publishing, 1994). Encapsulated fragrances according to the present invention preferably comprise fragrance ingredients selected from the group consisting of ADOXAL (2,6,10-trimethylundec-9-enal); AGRUMEX (2-(tert-butyl)cyclohexyl acetate); ALDEHYDE C 10 DECYLIC (decanal); ALDEHYDE C 11 MOA (2-methyldecanal); ALDEHYDE C 11 UNDECYLENIC (undec-10-enal); ALDEHYDE C 110 UNDECYLIC (undecanal); ALDEHYDE C 12 LAURIC (dodecanal); ALDEHYDE C 12 MNA PURE (2-methylundecanal); ALDEHYDE ISO C 11 ((E)-undec-9-enal); ALDEHYDE MANDARINE ((E)-dodec-2-enal); ALLYL AMYL GLYCOLATE (allyl 2-(isopentyloxy)acetate); ALLYL CYCLOHEXYL PROPIONATE (allyl 3-cyclohexylpropanoate); ALLYL OENANTHATE (allyl heptanoate); AMBER CORE (1-((2-(tert-butyl)cyclohexyl)oxy)butan-2-ol); AMBERMAX (1,3,4,5,6,7-hexahydro.beta.,1,1,5,5-pentamethyl-2H-2,4a-Methanonaphthalene-8-ethanol); AMYL SALICYLATE (pentyl 2-hydroxybenzoate); APHERMATE (1-(3,3-dimethylcyclohexyl)ethyl formate); BELAMBRE ((1R,2S,4R)-2′-isopropyl-1,7,7-trimethylspiro[bicyclo[2.2.1]heptane-2,4′[1,3]dioxan]); BIGARYL (8-(sec-butyl)-5,6,7,8-tetrahydroquinoline); BOISAMBRENE FORTE ((ethoxymethoxy)-cyclododecane); BOISIRIS ((1S,2R,5R)-2-ethoxy-2,6,6-trimethyl-9-methylene-bicyclo[3.3.1]nonane); BORNYL ACETATE ((2S,4S)-1,7,7-trimethylbicyclo[2.2.1]-heptan-2-yl acetate); BUTYL BUTYRO LACTATE (1-butoxy-1-oxopropan-2-yl butyrate); BUTYL CYCLOHEXYL ACETATE PARA (4-(tert-butyl)cyclohexyl acetate); CARYOPHYLLENE ((Z)-4,11,11-trimethyl-8-methylenebicyclo[7.2.0]-undec-4-ene); CASHMERAN (1,1,2,3,3-pentamethyl-2,3,6,7-tetrahydro-1H-inden-4(5H)-one); CASSYRANE (5-tert-butyl-2-methyl-5-propyl-2H-furan); CITRAL ((E)-3,7-dimethylocta-2,6-dienal); CITRAL LEMAROME N ((E)-3,7-dimethylocta-2,6-dienal); CITRATHAL R ((Z)-1,1-diethoxy-3,7-dimethylocta-2,6-diene); CITRONELLAL (3,7-dimethyloct-6-enal); CITRONELLOL (3,7-dimethyloct-6-en-1-ol); CITRONELLYL ACETATE (3,7-dimethyloct-6-en-1-yl acetate); CITRONELLYL FORMATE (3,7-dimethyloct-6-en-1-yl formate); CITRONELLYL NITRILE (3,7-dimethyloct-6-enenitrile); CITRONELLYL PROPIONATE (3,7-dimethyloct-6-en-1-yl propionate); CLONAL (dodecanenitrile); CORANOL (4-cyclohexyl-2-methylbutan-2-ol); COSMONE ((Z)-3-methylcyclotetradec-5-enone); CYCLAMEN ALDEHYDE (3-(4-isopropylphenyl)-2-methylpropanal); CYCLOGALBANATE (allyl 2-(cyclohexyloxy)acetate); CYCLOHEXYL SALICYLATE (cyclohexyl 2-hydroxybenzoate); CYCLOMYRAL (8,8-dimethyl-1,2,3,4,5,6,7,8-octahydronaphthalene-2-carbaldehyde); DAMASCENONE ((E)-1-(2,6,6-trimethylcyclohexa-1,3-dien-1-yl)but-2-en-1-one); DAMASCONE ALPHA ((E)-1-(2,6,6-trimethylcyclohex-2-en-1-yl)but-2-en-1-one); DAMASCONE DELTA ((E)-1-(2,6,6-trimethylcyclohex-3-en-1-yl)but-2-en-1-one); DECENAL-4-TRANS ((E)-dec-4-enal); DELPHONE (2-pentylcyclopentanone); DIHYDRO ANETHOLE (propanedioic acid 1-(1-(3,3-dimethylcyclohexyl)ethyl) 3-ethyl ester); DIHYDRO JASMONE (3-methyl pentylcyclopent-2-enone); DIMETHYL BENZYL CARBINOL (2-methyl-1-phenylpropan ol); DIMETHYL BENZYL CARBINYL ACETATE (2-methyl-1-phenylpropan-2-yl acetate); DIMETHYL BENZYL CARBINYL BUTYRATE (2-methyl-1-phenylpropan-2-yl butyrate); DIMETHYL OCTENONE (4,7-dimethyloct-6-en-3-one); DIMETOL (2,6-dimethylheptan ol); DIPENTENE (1-methyl-4-(prop-1-en-2-yl)cyclohex-1-ene); DUPICAL ((E) ((3aS,7aS)-hexahydro-1H-4,7-methanoinden-5(6H)-ylidene)butanal); EBANOL ((E) methyl-5-(2,2,3-trimethylcyclopent-3-en-1-yl)pent-4-en-2-ol); ETHYL CAPROATE (ethyl hexanoate); ETHYL CAPRYLATE (ethyl octanoate); ETHYL LINALOOL ((E)-3,7-dimethylnona-1,6-dien-3-ol); ETHYL LINALYL ACETATE ((Z)-3,7-dimethylnona-1,6-dien-3-yl acetate); ETHYL OENANTHATE (ethyl heptanoate); ETHYL SAFRANATE (ethyl 2,6,6-trimethylcyclohexa-1,3-diene-1-carboxylate); EUCALYPTOL ((1s,4s)-1,3,3-trimethyl-2-oxabicyclo[2.2.2]octane); FENCHYL ACETATE ((2S)-1,3,3-trimethylbicyclo[2.2.1]heptan-2-yl acetate); FENCHYL ALCOHOL ((1S,2R,4R)-1,3,3-trimethylbicyclo[2.2.1]heptan-2-ol); FIXOLIDE (1-(3,5,5,6,8,8-hexamethyl-5,6,7,8-tetrahydronaphthalen-2-yl)ethanone); FLOR-ALOZONE (3-(4-ethylphenyl)-2,2-dimethylpropanal); FLORHYDRAL (3-(3-isopropylphenyl)butanal); FLOROCYCLENE ((3aR,6S,7aS)-3a,4,5,6,7,7a-hexahydro-1H4,7-methanoinden-6-yl propionate); FLOROPAL (2,4,6-trimethyl-4-phenyl-1,3-dioxane); FRESKOMENTHE (2-(sec-butyl)cyclohexanone); FRUITATE ((3aS,4S,7R,7aS)-ethyl octahydro-1H-4,7-methanoindene-3a-carboxylate); FRUTONILE (2-methyldecanenitrile); GALBANONE PURE (1-(3,3-dimethylcyclohex-1-en-1-yl)pent-4-en-1-one); GARDOCYCLENE ((3aR,6S,7aS)-3a,4,5,6,7,7a-hexahydro-1H-4,7-methanoinden-6-yl isobutyrate); GERANIOL ((E)-3,7-dimethylocta-2,6-dien-1-ol); GERANYL ACETATE SYNTHETIC ((E)-3,7-dimethylocta-2,6-dien-1-yl acetate); GERANYL ISOBUTYRATE ((E)-3,7-dimethylocta-2,6-dien-1-yl isobutyrate); GIVESCONE (ethyl 2-ethyl-6,6-dimethylcyclohex-2-enecarboxylate); HABANOLIDE ((E)-oxacyclohexadec-12-en-2-one); HEDIONE (methyl 3-oxo-2-pentylcyclopentaneacetate); HERBANATE ((2S)-ethyl 3-isopropylbicyclo[2.2.1]hept-5-ene-2-carboxylate); HEXENYL-3-CIS BUTYRATE ((Z)-hex-3-en-1-yl butyrate); HEXYL CINNAMIC ALDEHYDE ((E)-2-benzylideneoctanal); HEXYL ISOBUTYRATE (hexyl isobutyrate); HEXYL SALICYLATE (hexyl 2-hydroxybenzoate); INDOFLOR (4,4a,5,9b-tetrahydroindeno[1,2-d][1,3]dioxine); IONONE BETA ((E)-4-(2,6,6-trimethylcyclohex-1-en-1-yl)but-3-en-2-one); IRISONE ALPHA ((E)-4-(2,6,6-trimethylcyclohex-2-en-1-yl)but-3-en-2-one); IRONE ALPHA ((E)-4-(2,5,6,6-tetramethylcyclohex-2-en-1-yl)but-3-en-2-one); ISO E SUPER (1-(2,3,8,8-tetramethyl-1,2,3,4,5,6,7,8-octahydronaphthalen-2-yl)ethanone); ISOCYCLO-CITRAL (2,4,6-trimethylcyclohex-3-enecarbaldehyde); ISONONYL ACETATE (3,5,5-trimethylhexyl acetate); ISOPROPYL METHYL-2-BUTYRATE (isopropyl 2-methyl butanoate); ISORALDEINE 70 ((E)-3-methyl-4-(2,6,6-trimethylcyclohex-2-en-1-yl)but-3-en-2-one); JASMACYCLENE ((3aR,6S,7aS)-3a,4,5,6,7,7a-hexahydro-1H-4,7-methanoinden-6-yl acetate); JASMONE CIS ((Z)-3-methyl-2-(pent-2-en-1-yl)cyclopent-2-enone); KARANAL (5-(sec-butyl)-2-(2,4-dimethylcyclohex-3-en-1-yl)-5-methyl-1,3-dioxane); KOAVON E ((Z)-3,4,5,6,6-pentamethylhept-3-en-2-one); LEAF ACETAL ((Z)-1-(1-ethoxyethoxy)hex-3-ene); LEMONILE ((2E,6Z)-3,7-dimethylnona-2,6-dienenitrile); LIFFAROME GIV ((Z)-hex-3-en-1-yl methyl carbonate); LILIAL (3-(4-(tert-butyl)phenyl)-2-methylpropanal); LINALOOL (3,7-dimethylocta-1,6-dien-3-ol); LINALYL ACETATE (3,7-dimethylocta-1,6-dien-3-yl acetate); MAHONIAL ((4E)-9-hydroxy-5,9-dimethyl-4-decenal); MALTYL ISOBUTYRATE (2-methyl-4-oxo-4H-pyran-3-yl isobutyrate); MANZANATE (ethyl 2-methylpentanoate); MELONAL (2,6-dimethylhept-5-enal); MENTHOL (2-isopropyl-5-methylcyclohexanol); MENTHONE (2-isopropyl-5-methylcyclohexanone); METHYL CEDRYL KETONE (1-((1S,8aS)-1,4,4,6-tetra methyl-2,3,3a,4,5,8-hexahydro-1H-5,8a-methanoazulen-7-yl)ethanone); METHYL NONYL KETONE EXTRA (undecan-2-one); METHYL OCTYNE CARBONATE (methyl non-2-ynoate); METHYL PAMPLEMOUSSE (6,6-dimethoxy-2,5,5-trimethylhex-2-ene); MYRALDENE (4-(4-methylpent-3-en-1-yl)cyclohex-3-enecarbaldehyde); NECTARYL (2-(2-(4-methylcyclohex-3-en-1-yl)propyl)cyclopentanone); NEOBERGAMATE FORTE (2-methyl-6-methyleneoct-7-en-2-yl acetate); NEOFOLIONE ((E)-methyl non-2-enoate); NEROLIDYLE ((Z)-3,7,11-trimethyldodeca-1,6,10-trien-3-yl acetate); NERYL ACETATE HC ((Z)-3,7-dimethylocta-2,6-dien-1-yl acetate); NONADYL (6,8-dimethylnonan-2-ol); NONENAL-6-CIS ((Z)-non-6-enal); NYMPHEAL (3-(4-isobutyl-2-methylphenyl)propanal); ORIVONE (4-(tertpentyl)cyclohexanone); PARADISAMIDE (2-ethyl-N-methyl-N-(m-tolyl)butanamide); PELARGENE (2-methyl-4-methylene-6-phenyltetrahydro-2H-pyran); PEONILE (2-cyclohexylidene-2-phenylacetonitrile); PETALIA (2-cyclohexylidene-2-(otolyl)acetonitrile); PIVAROSE (2,2-dimethyl-2-pheylethyl propanoate); PRECYCLEMONE B (1-methyl-4-(4-methylpent-3-en-1-yl)cyclohex-3-enecarbaldehyde); PYRALONE (6-(sec-butyl)quinoline); RADJANOL SUPER ((E)-2-ethyl-4-(2,2,3-trimethylcyclopent-3-en-1-yl)but-2-en-1-ol); RASPBERRY KETONE (N112) (4-(4-hydroxyphenyl)butan-2-one); RHUBAFURANE (2,2,5-trimethyl-5-pentylcyclopentanone); ROSACETOL (2,2,2-trichloro-1-phenylethyl acetate); ROSALVA (dec-9-en-1-ol); ROSYFOLIA ((1-methyl-2-(5-methylhex-4-en-2-yl)cyclopropyl)-methanol); ROSYRANE SUPER (4-methylene phenyltetrahydro-2H-pyran); SERENOLIDE (2-(1-(3,3-dimethylcyclohexyl)-ethoxy) methylpropyl cyclopropanecarboxylate); SILVIAL (3-(4-isobutylphenyl) methylpropanal); SPIROGALBANONE (1-(spiro[4.5]dec-6-en-7-yl)pent-4-en-1-one); STEMONE ((E)-5-methylheptan-3-one oxime); SUPER MUGUET ((E)-6-ethyl-3-methyloct-6-en-1-ol); SYLKOLIDE ((E)-2-((3,5-dimethylhex-3-en-2-yl)oxy)-2-methylpropyl cyclopropanecarboxylate); TERPINENE GAMMA (1-methyl-4-propan-2-ylcyclohexa-1,4-diene); TERPINOLENE (1-methyl-4-(propan-2-ylidene)cyclohex-1-ene); TERPINYL ACETATE (2-(4-methylcyclohex-3-en-1-yl)propan-2-yl acetate); TETRAHYDRO LINALOOL (3,7-dimethyloctan-3-ol); TETRAHYDRO MYRCENOL (2,6-dimethyloctan-2-ol); THIBETOLIDE (oxacyclohexadecan-2-one); TRIDECENE-2-NITRILE ((E)-tridec-2-enenitrile); UNDECAVERTOL ((E)-4-methyldec-3-en-5-ol); VELOUTONE (2,2,5-trimethyl-5-pentylcyclopentanone) and VIRIDINE ((2,2-dimethoxyethyl)benzene); ZINARINE (2-(2,4-dimethylcyclohexyl)pyridine).

The shell of the microcapsules comprises encapsulating materials which are preferably impervious to the functional core material when the microcapsules are stored in the consumer product. Such impervious shell materials are well known to the art.

The shell may comprise a material selected from the group consisting of aminoplast resins, such as melamine-formaldehyde resins, urea-formaldehyde resins and melamine-urea-formaldehyde resins, polyurea resins, poly(meth)acrylate resins, polyester resins, polysaccharides, proteins, polypeptides, silicon oxides and organosiloxanes.

Such microcapsules may be obtained by any method known to the art. The size of the microcapsules obtained by such methods may be optimized to the desired value by varying the stirring speed and/or stirrer geometry during the synthesis of the microcapsules, in particular during the emulsification step preceding the formation of the microcapsule shell.

For example, aminoplast microcapsules may be obtained as described in EP 2 111 214 B1, Example 3, sample P4.1; US 2018/0185808 A1, Example 1; WO 2016/207187 A1, Examples 1, 2 and 2b. For example, polyurea microcapsules may be obtained as described in WO 2011/160733 A1, Example 2.

These microcapsules are usually obtained in the form of slurries, meaning dispersions or suspensions of microcapsules in an aqueous phase. Typically, these slurries may comprise from 30 to 50 wt.-% microcapsules, preferably from 35 to 45 wt.-%. The percentage of microcapsules in a slurry is referred to as “solid content” and is measured experimentally by methods known to the art. The solid content of a slurry includes both core and shell materials. For example, the solid content may be measured by using a thermo-balance operating at, for example, 120° C. The solid content, expressed as weight percentage of the initial slurry deposited on the balance may be taken at the point where the drying-induced rate of weight change has dropped below 0.1%/min.

Preferably, the microcapsules are stabilized against agglomeration, creaming or sedimentation by employing a suspending agent. The suspending agent may be added under stirring before the microcapsules are formed, during formation of the microcapsules or after the microcapsules have been formed (post-addition). Preferably, the suspending agent is post-added to the slurry of microcapsules under stirring.

Suspending agents may be selected from a broad class of water-soluble or water-dispersible polymers having anionic, cationic, zwitterionic or non-ionic character. Water-soluble or water-dispersible polymers useful for the sake of the present invention encompass:

Polysaccharides, such as starch, modified starch, dextrin, maltodextrin, and cellulose derivatives, and their quaternized forms; natural gums such as alginate esters, carrageenan, xanthan, agar-agar, pectins, pectic acid, and natural gums such as gum arabic, gum tragacanth and gum karaya, guar gums and quaternized guar gums; gelatine, protein hydrolysates and their quaternized forms; synthetic polymers and copolymers, such as poly(vinyl pyrrolidone-co-vinyl acetate), poly(vinyl alcohol-co-vinyl acetate), poly((met)acrylic acid), poly(maleic acid), poly(alkyl(meth)acrylate-co-(meth)acrylic acid), poly(acrylic acid-co-maleic acid)copolymer, poly(alkyleneoxide), poly(vinyl-co-methylether), poly(vinylether-co-maleic anhydride), and the like, as well as poly(ethyleneimine), poly((meth)acrylamide), poly(alkyleneoxide-co-dimethylsiloxane), poly(amino dimethylsiloxane), and their quaternized forms.

In particular embodiments of the present invention, the consumer product may be selected from the group consisting of liquid detergents, hard surface cleaners, shampoos, shower gels, liquid soaps, dish washing liquids and fragranced products comprising ethanol.

Liquid detergent compositions that are concerned by the invention may be regular or concentrated detergents. They may be available as single dose “pouches” or “liquid tabs”.

Laundry care liquid detergents comprise anionic and/or non-ionic surfactants, and mixtures thereof. Typical anionic surfactants include sodium lauryl sulphate, sodium laureth sulphate, sodium trideceth sulphate, ammonium lauryl sulphate, ammonium laureth sulphate, potassium laureth sulphate, linear alkyl benzene sulfonates, sodium tridecyl benzene sulfonate, sodium dodecyl benzene sulfonate, sodium xylene sulfonate, monoethanolamine lauryl sulphate, monoethanolamine laureth sulphate, triethanolamine lauryl sulphate, triethanolamine laureth sulphate, lauryl sarcosine, cocoyl sarcosine, sodium lauryl sarcosinate, sodium lauroyl sarcosinate, triethylamine lauryl sulphate, triethylamine laureth sulphate, diethanolamine lauryl sulphate, diethanolamine laureth sulphate, lauric monoglyceride sodium sulphate, ammonium cocoyl sulphate, ammonium lauroyl sulphate, sodium cocoyl sulphate, sodium lauroyl sulphate, sodium cocoyl isethionate, potassium cocoyl sulphate, potassium lauryl sulphate, monoethanolamine cocoyl sulphate, monoethanolamine lauryl sulphate, triethanolamine lauryl sulphate, C5-C17 acyl-N-(C1-C4 alkyl) glucamine sulphate, C5-C17 acyl-N-(C1-C4 hydroxyalkyl) glucamine sulphate, sodium hydroxyethyl-2-decyl ether sulphates, sodium methyl-2-hydroxydecyl ether sulphates, sodium hydroxyethyl-2-dodecyl ether sulphates, sodium monoethoxylated lauryl alkyl sulphates, C12-C18 alkyl sulfonates, ethoxylated or native linear and ramified C12-C18 alcohol sulphates, ethoxylated or native linear and ramified C12-C18 alcohol sulphates, and mixtures thereof. The above-mentioned anionic surfactants may also be used in their unneutralized, acid form. Typically, the level of anionic surfactants in liquid detergents is from 1 to 40% by weight, more particularly from 5 to 35% by weight of the liquid detergent.

Typical non-ionic surfactants include C6-C24 alkyl ethoxylates with 1 to 12 ethylene oxide units. The alkyl chain of the aliphatic alcohol can either be straight or branched, primary or secondary, and generally contains from 6 to 22 carbon atoms. Further examples of non-ionic surfactants include the condensation products of fatty acids with glucamines, such as C12-C16 akyl N-methyl glucamide, and/or the condensation product of fatty acids with eth-oxylated amines; C10-C20 alkyl mono- or dialkanolamides, where the alkyloxy group has 1 to 3 carbon atoms, C.10-C20 alkyl mono- or di-alknolamide having an intermediate polyoxyalkylene moiety having 2 to 20 alkyleneoxide groups between the alkyl moiety and the alkanolamide moiety; alkyl amidopropyl dimethylamine; fatty acid alkyl esters, such as sorbitol esters with oleic, myristic, stearic, palmitic acid, and the like, also known under the trade name Tween, such as Tween 20, Tween 40, and Tween 60; alkyl polyglycosides including, for example, C8-C10 alkyl polyglycosides, C12-C16 alkyl polyglycosides, C5 amyl polyglycosides. Further non-ionic surfactants include glycerol-based surfactants, such as fatty acid polyglyceryl esters like octanoic acid hexaglyceryl ester, decanoic acid tetraglyceryl ester, riccinoleic acid hexaglyceryl ester and cocoic acids tetraglyceryl esters and their mixtures. The term “alkyl” as used hereinabove for the non-ionic sugar-based surfactant refers to saturated linear alkyl residues having 3 to 21 carbon atoms, including hexyl, octyl, decanyl, dodecanyl, tetradecanyl, hexadecanyl, and octadecanyl. Typically, the level of non-ionic surfactants in liquid detergents is from 0 to 40% by weight, more particularly from 10 to 35% by weight of the detergent.

In some cases, the liquid detergent may also comprise cationic, cationogenic, zwitterionic and/or amphoteric surfactants.

Unit dose, pouched formats are well known in the art. Pouched formats typically comprise a liquid detergent base surrounded by a water-soluble film. Owing to the fact that these liquid detergent compositions are contained within a water-soluble or dispersible film, they are characterized by high levels of surfactants and very low concentrations of water.

Preferred water-soluble films are polymers and co-polymers based on polyvinyl alcohol and thermoplastic starch derivatives, wherein polyvinyl alcohol-based polymers are the most often used. Liquid detergent composition that can be held in water-soluble pouches may typically comprise water, solvents, bleaching agents, enzymes, enzyme stabilizing systems, chelating agents, surfactants, neutralizing agents, builders, fillers, anti-redeposition or soil dispersing polymers, fabric caring or enhancing polymers, dye transfer inhibitors, flocculating, deflocculating and thickening agents and fabric softening agents. Such compositions contain preferably less than 0.2% of borate ions but preferably essentially free of borate or perborate.

The level of water in the liquid detergent composition unit dose format is such that the water-soluble polymer forming the pouch does not dissolve as a result of contact with the composition. The level of water in the liquid detergent composition is less than 50% by weight, more particularly less than 20% by weight, still more particularly less than 10% by weight, and may be even as low as 5% by weight of the liquid detergent composition.

Neutralizing agents that may be employed in detergent compositions are preferably selected from an organic bases such as amines, e.g. mono-ethanolamine, triethanolamine, organic Lewis bases, and mixtures thereof, but inorganic bases, such as sodium hydroxide, potassium hydroxide and ammonium hydroxide can also be used. The level of neutralizing agent in the composition is typically from 5 to 15% by weight of the liquid detergent composition.

Preferred solvents are those solvents which do not dissolve a water-soluble polymer forming a pouch. These solvents may have a low polarity or a high polarity. Low polarity solvents include typically linear and/or branched paraffin hydrocarbons. High polarity water-soluble or partially soluble or water miscible solvents include typically alcohols, such as methanol, ethanol, propanol, isopropanol, butanol, diols, such as 1,2-propanediol, 1,3-propanediol, glycerol, sorbitol, 2-amino-2-ethyl propanol, ethers, polyethers, short chain di-, tri-N-substituted alkylamines, short chain alkyl amides, short chain alkyl carboxylic acid lower alkyl esters, ketones, such as short chain alkyl ketones, including acetone. The liquid composition may comprise from 10 to 70% by weight of water-soluble solvents.

All-purpose cleaners comprise typically from 0.1 to 25% by weight or, preferably from 2 to 20% by weight of anionic and non-ionic surfactant, preferably selected from, but not limited to sodium alkyl sulfonates and alkyl ethoxylates; from 1 to 10% by weight, preferably from 2 to 6% by weight soaps, for sample sodium fatty acid carboxylates; from 1 to 15% by weight, preferably from 2 to 10% by weight of alkalinity sources, for example sodium carbonate; from 1 to 10% by weight inorganic builders, for example sodium citrate-citric acid mixture; from 0 to 2% by weight organic builders, for example sodium polycarboxylate; from 0.0001 to 0.5% by weight, preferably from 0.0003 to 0.1% by weight of one or more preservatives; and, up to 5% by weight of one or more water-soluble solvents, citric acid, triethanolamine, sodium hydroxide, potassium hydroxide, ammonia and/or oils.

Shampoos comprise typically from 3% to 25% by weight, for example from 12% to 20% by weight or from 14% to 18% by weight of one or more anionic surfactants; from 0.5% to 20% by weight, for example from 1% to 10% by weight of zwitterionic and/or amphoteric surfactants; from 0% to 10% by weight on non-ionic surfactants; from 20% to 90% by weight of an aqueous phase, comprising optionally water-soluble solvents; from 0.0001 to 0.5% by weight, preferably from 0.0003 to 0.1% by weight of one or more preservatives and optionally benefit agents, such as moisturizers, emollients, thickeners, anti-dandruff agents, hair growth promoting agents, vitamins, nutrients, dyes and hair colorants.

Fabric conditioners or softeners typically comprise nitrogen-containing cationic surfactants having one or two alkyl chains comprising 16 to 22 carbon atoms and optionally hydroxyl groups. The cationic group is preferably a quaternary ammonium, an imidazolium or an amido salt. The quaternary ammonium group has additionally two to three alkyl groups having 1 to 4 carbon or hydroxyalkyl, hydroxyl groups or alkoxy groups, having typically 1 to 10 ethylene oxide moieties, and an anion selected from the group of halides, hydroxides, acetates and methylsulfate. The long alkyl chain is preferably bound to the cationic group via an ester group. Typical examples of such fabric conditioning actives include esterquat (N-methyl-N,N,bis[2-(C16-C18-acetoxy)ethyl)]-N-(2-hydroxyethyl) ammonium methosulfate), diesterquat (N,N,N-trimethyl-N-[1,2-di-(C16-C18-acyloxy)propyl ammonium salts), DEEDMAC (N,N-dimethyl-N,N-bis([2-(−[(1-oxooctadecyl)oxy]ethyl) ammonium chloride, HEQ (N,N,N-trimethyl-N-[(2)-2-hydroxy-3-[(1-oxo-octadec-9-enyl)oxy]] ammonium chloride, TEAQ (diquaternized methylsulfate salt of the reaction product between C10-C20 saturated and unsaturated fatty acids and triethanolamine), glycerine-based polyol esterquats, ethyl-tallowalkyl imidazolinium methyl sulphate, ditallowalkyl dimethylammonium methyl sulfate, methyl tallowalkyl amido ethyl tallowalkyl imidazolinium methyl sulfate, b-hydroxyethyl ethylenediamine erivatives, polyammonium and the like, and mixture thereof.

Typical non-ionic surfactants that may be present in fabric conditioners or softeners include, but are not limited to alkyl and alkylbenzyl alcohol alkoxylates or polyalkoxylated carboxylic acids, polyalkoxylated amines, polyalkoxylated glycol or glycerol esters, polyalkoxylated sorbitan esters or alkanoamides.

Hair conditioners typically comprise nitrogen-containing cationic surfactants, such as alkyl quaternary ammonium salts, for example cetrimonium chloride or trimethyl stearyl ammonium chloride; and cationic polymers, such as quaternary nitrogen-substituted cellulose ether derivatives, quaternary nitrogen-containing poly(trialkylaminoethyl methacrylate) derivatives, quaternary nitrogen-containing poly(vinylpyrrolidone), and cyclic cation group-containing polymers such as a diallyl quaternary ammonium homopolymer and a diallyl quaternary ammonium copolymer. Hair conditioners also comprise zwitterionic surfactants and betaines, such as cocamidopropyl betaine; anionic surfactant, such as anionic surface active agent, including carboxylic acid salt-type, sulfonic acid salt-type and sulfuric acid ester salt-type anionic surface active agents, more particularly N-acylaminocarboxylic acid salt-type and ether carboxylic acid salt-type surface active agents; fatty alcohols, such as cetyl alcohol, stearyl alcohol, behenyl alcohol, isostearyl alcohol, octyldodecanol and oleyl alcohol; oils, such as petrolatum and vegetable oils; amodimethicone and silicone polymers, such as methylpolysiloxane, polyoxyethylene-methylpolysiloxane, polyoxypropylenemethylpolyoxysiloxane, poly(oxyethylene, oxypropylene) methylpolysiloxane, methylphenylpolysiloxane, fatty acid-modified polysiloxane, fatty acid alcohol-modified polysiloxane and amino acid-modified polysiloxane; wetting agents such as ethylene glycol, propylene glycol, 1,3-butylene glycol, glycerol and sorbitol; emulsifying agents such as glycerol monostearate and polyoxyethylene sorbitan monolaurate; hydrocarbons such as liquid paraffin, Vaseline and squalene; esters such as isoproidyl myristate and octyldodecyl myristate; cellulose derivatives such as hydroxyethyl cellulose, hydroxypropyl cellulose and carboxymethyl cellulose; and anionic polymers such as acrylic acid-type polymers.

In a second aspect, the present invention provides a method for obtaining a consumer product, in particular a consumer product as described herein above. The method comprises the steps of:
a) Selecting a transparent or translucent product base;
b) Adding a plurality of microcapsules in the form of a slurry, so that the total surface area of the plurality of microcapsules comprised in one litre of consumer product is from 0.02 to 0.27 m², preferably from 0.02 to 0.12 m²;
c) Dispersing the plurality of the microcapsules.

With regard to step c), the action of dispersing the plurality of the microcapsules may be performed by applying suitable dispersive shear forces by any mean known to the art, such as for example a propeller, a blade mixer, a dissolver, a rotor-stator mixer, a low pressure homogenizer or a static mixer.

In a third aspect, the present invention provides a use of a plurality of microcapsules in a consumer product as described herein above.

Further features and particular advantages of the present invention become apparent from the following examples.

### EXAMPLE 1—FORMATION OF AMINOPLAST MICROCAPSULES HAVING VARIOUS DIAMETERS

The microcapsules have been obtained by performing the method disclosed in US 2018/0185808 A1, Example 1. The volume median diameter of the microcapsules was varied by varying the stirring speed during emulsification step 3. In the present case, the volume of the reactor was 0.3 L and the stirrer was a cross-beam stirrer with pitched beam.

The volume median diameter Dᵥ(50) and the surface mean diameter D(3,2) of the microcapsules were measured using a Malvern Mastersizer 2000S Particle Size Analyzer. A few drops of slurry were added to a circulating stream of degassed water flowing through a scattering cell. Under such conditions of dilution, the angular distribution of the scattering intensity was measured and analyzed by using the proprietary software provided with the apparatus to provide the size distribution of the droplets present in the sample, form which the surface mean and volume median diameters were obtained.

The solid content of the slurries was 41 to 43 wt.-%.

In these examples, the density of the microcapsules was adjusted to 1.04 to 1.06 g/cm³ at 25° C.

TABLE 1
Volume median diameter and surface average diameter of the
microcapsules in the different slurries obtained in Example 1

| Example | Dᵥ (50) [μm] | D(3,2) [μm] |
|---|---|---|
| Example 1.1 | 6 | 7 |
| Example 1.2 | 11 | 12 |
| Example 1.3 | 30 | 39 |
| Example 1.4 | 66 | 53 |

### EXAMPLE 2—FORMATION OF LIQUID DETERGENT SAMPLES COMPRISING MICROCAPSULES

A series of translucent or transparent liquid fabric care detergent samples (HDLD) were prepared by admixing different amounts of the microcapsule slurries obtained in Example 1 with a regular, transparent unperfumed base having a density of 1.05 g/cm³ at 25° C. (see Table 2). The effective amounts of microcapsules (m_{caps}) were obtained from the amounts of added slurries, based on the measured solid content of these slurries. The mean total surface area of the microcapsules in each sample was calculated by applying Equation 6, taking a value of 1.05 g/cm³ for the density of the microcapsules (ρ_{caps}).

The turbidity of the samples was measured at 25° C. by using a HACH 2100Q iS turbidimeter. The value returned by the instrument was the ratio of the scattered light intensity, measured at an angle of 90° with respect to the direction of the incident light, divided by the transmitted light intensity, measured at an angle of 180 with respect to the direction of the incident light. The turbidity values are given in Nephelometric Turbidity Unit (NTU). The turbidity values are reported in Table 2.

The olfactive performance of the samples was evaluated as follows: 75 g of the laundry liquid detergent was used in a side-loaded wash machine (20 L capacity, loaded with 1 kg terry towels); a wash cycle was performed at a temperature of 40° C., followed by spin-drying. The terry towels for 24 hours at room temperature. The evaluation was performed by gently rubbing one part of the terry towel on another part of same terry towel. The olfactive performance (intensity) was assessed by a panel of 4 experts and rated on a scale of 1-5 (1=barely noticeable, 2=weak, 3=medium, 4=strong and 5=very strong).

TABLE 2
Characteristics of liquid fabric care detergent comprising
different microcapsules at different concentration

| | Capsules | Mean total | | |
|---|---|---|---|---|
| | amount | surface area of | Turbidity | Olfactive |
| Slurry | [g/l] | the capsules [m²/l] | [NTU] | Performance |
| Example 1.1.1 | 0.01 | 0.014 | 52 | 0 |
| Example 1.1.2 | 0.21 | 0.28 | 580 | 0.5 |
| Example 1.2.1 | 0.21 | 0.13 | 237 | 1 |
| Example 1.2.2 | 0.37 | 0.27 | 267 | 2 |
| Example 1.2.3 | 0.84 | 0.53 | 480 | 2 |
| Example 1.3.1 | 0.21 | 0.068 | 83 | 1 |
| Example 1.3.2 | 0.42 | 0.14 | 152 | 2 |
| Example 1.3.3 | 0.84 | 0.27 | 232 | 2.5 |
| Example 1.3.4 | 1.26 | 0.41 | 360 | 2.5 |
| Example 1.4.1 | 0.42 | 0.02 | 37 | 1 |
| Example 1.4.2 | 0.84 | 0.05 | 58 | 1 |
| Example 1.4.3 | 1.26 | 0.070 | 75 | 1 |

As apparent from Table 2, samples having a mean total surface area of microcapsules comprised in one litre of consumer product from 0.02 to 0.27 m² are translucent or even transparent, while still having good olfactive performance. On the other hand, samples falling out of this range are either turbid or show insufficient olfactive performance. Furthermore, samples having a mean total surface area of microcapsules comprised in one litre of consumer product from 0.02 to 0.12 m² show particularly low turbidity values, while still having good olfactive performance.

1. A consumer product comprising a plurality of microcapsules, the microcapsules comprising a core and a shell around the core, the core comprising at least one functional material, wherein the mean total surface area of the plurality of microcapsules comprised in one litre of consumer product is from 0.02 to 0.27 m².

2. The consumer product according to claim 1, wherein the mean total surface area of the plurality of microcapsules is calculated based on the surface mean diameter D(3,2) and on the volume median diameter Dᵥ(50) of the microcapsules.

3. The consumer product according to claim 1, wherein the volume median diameter Dᵥ(50) is from 5 to 50 μm.

4. The consumer product according to claim 1, wherein the consumer product has a turbidity lower than 350 NTU at 25° C.

5. The consumer product according to claim 1; wherein the at least one functional material is selected from a fragrance, a flavour and a cosmetic ingredient.

6. The consumer product according to claim 5, wherein the fragrance comprises at least one fragrance ingredient selected from the group consisting of: 2,6,10-trimethylundec-9-enal; 2-(tert-butyl)cyclohexyl acetate; decanal; 2-methyldecanal; undec-10-enal); undecanal; dodecanal; 2-methylundecanal; (E)-undec enal; (E)-dodec-2-enal; allyl 2-(isopentyloxy)acetate; allyl 3-cyclohexylpropanoate; allyl heptanoate; 1-((2-(tert-butyl)cyclohexyl)oxy)-butan-2-ol; 1,3,4,5,6,7-hexahydro.beta.,1,1,5,5-pentamethyl-2H-2,4a-methanonaphthalene-8-ethanol; pentyl 2-hydroxybenzoate; 1-(3,3-dimethylcyclohexyl)ethyl formate; (1R,2S,4R)-2T-isopropyl-1,7,7-tri methyl-spiro[bicyclo[2.2.1]heptane-2,4′[1,3]dioxane]; 8-(sec-butyl)-5,6,7,8-tetra-hydroquinoline); (ethoxymethoxy)-cyclododeca ne; (1S,2R,5R)-2-ethoxy-2,6,6-trimethyl-9-methylene-bicyclo[3.3.1]nonane; (2S,4S)-1,7,74n methyl-bicyclo[2.21]-heptan-2-yl acetate; 1-butoxy-1-oxopropan-2-yl butyrate; 4-(tert-butyl)cyclohexyl acetate; (Z)-4,11,11-trimethyl-8-methylene-bicyclo[7.2.0]-undec-4-ene; 1,1,2,3,3-pentamethyl-2,3,6,7-tetrahydro-1H-inden-4(5H)-one; 5-tert-butyl-2-methyl-5-propyl-2H-furan; (E)-3,7-dimethylocta-2,6-dienal; (E)-3,7-dimethylocta-2,6-dienal; (Z)-1,1-diethoxy-3,7-dimethylocta-2,6-diene; 3,7-dimethyloct-6-enal; 3,7-dimethyloct-6-en-1-ol 3,7-dimethyloct-6-en-1-yl acetate; 3,7-dimethyloct-6-en-1-yl formate; 3,7-dimethyloct-6-enenitrile; 3,7-dimethyloct-6-en-1-yl propionate; dodecanenitrile; 4-cyclohexyl-2-methylbutan-2-ol; (Z)-3-methylcyclotetradec-5-enone; 3-(4-isopropylphenyl)-2-methylpropanal; (allyl 2-(cyclohexyloxy)acetate; cyclohexyl 2-hydroxybenzoate; 8,8-dimethyl-1,2,3,4,5,6,7,8-octahydronaphthalene-2-carbaldehyde; (E)-1-(2,6,6-trimethylcyclohexa-1,3-dien-1-yl)but-2-en-1-one; (E)-1-(2,6,6-trimethylcyclohex-2-en-1-yl)but-2-en-1-one; (E)-1-(2,6,6-trimethyl-cyclohex-3-en-1-yl)but-2-en-1-one; (E)-dec-4-enal; 2-pentylcyclopentanone; propanedioic acid 1-(1-(3,3-dimethylcyclohexyl)ethyl) 3-ethyl ester; 3-methyl-2-pentylcyclopent-2-enone; 2-methyl-1-phenylpropan-2-ol; 2-methyl-1-phenylpropan-2-yl acetate; 2-methyl-1-phenylpropan-2-yl butyrate; 4,7-dimethyloct-6-en-3-one; 2,6-dimethylheptan-2-ol; 1-methyl-4-(prop-1-en-2-yl)cyclohex-1-ene; (E)-4-((3aS,7aS)-hexahydro-1H-4,7-methanoinden-5(6H)-ylidene)butanal; (E) methyl-5-(2,2,3-trimethylcyclopent-3-en-1-yl)pent-4-en-2-ol; ethyl hexanoate; ethyl octanoate; (E)-3,7-dimethylnona-1,6-dien-3-ol; (Z)-3,7-dimethyl nona-1,6-dien-3-yl acetate; ethyl heptanoate; ethyl 2,6,6-trimethylcyclohexa-1,3-diene-1-carboxylate; (1s,4s)-1,3,3-trimethyl-2-oxa-bicyclo[2.2.2]octane; (2S)-1,3,3-trimethylbicyclo[2.2.1]heptan-2-yl acetate; (1S,2R,4R)-1,3,3-trimethylbicyclo[2.2.1]heptan-2-ol); 1-(3,5,5,6,8,8-hexa-methyl-5,6,7,8-tetrahydronaphthalen-2-yl)ethanone; 3-(4-ethylphenyl)-2,2-dimethylpropanal; 3-(3-isopropylphenyl)butanal; (3a R,6S,7aS)-3a,4,5,6,7,7a-hexahydro-1H-4,7-methanoinden-6-yl propionate; 2,4,6-trimethyl-4-phenyl-1,3-dioxane; 2-(sec-butyl)cyclohexanone); (3aS,4S,7R,7aS)-ethyl octa hydro-1H-4,7-methanoindene-3a-carboxylate; 2-methyldecanenitrile; 1-(3,3-dimethylcyclohex-1-en-1-yl)pent-4-en-1-one; (3aR,6S,7aS)-3a,4,5,6,7,7a-hexahydro-1H-4,7-methanoinden-6-yl isobutyrate; (E)-3,7-dimethylocta-2,6-dien-1-ol; (E)-3,7-dimethylocta-2,6-dien-1-yl acetate; (E)-3,7-dimethylocta-2,6-2-one; 1-(2,3,8,8-tetramethyl-1,2,3,4,5,6,7,8-octahydronaphthalen-2-yl)ethanone); 2,4,6-trimethylcyclohex-3-enecarbaldehyde; 3,5,5-dien-1-yl isobutyrate; ethyl 2-ethyl-6,6-dimethylcyclohex-2-enecarboxylate; (E)-oxacyclohexadec-12-en-2-one; methyl 3-oxo-2-pentylcyclopentaneacetate; (2S)-ethyl 3-isopropylbicyclo[2.2.1]hept-5-ene-2-carboxylate; (Z)-hex-3-en-1-yl butyrate; (E)-2-benzylideneoctanal; hexyl isobutyrate; hexyl 2-hydroxybenzoate; 4,4a,5,9b-tetrahydroindeno[1,2-d][1,3]dioxine; (E)-4-(2,6,6-trimethylcyclohex-1-en-1-yl)but-3-en-2-one; (E)-4-(2,6,6-tri methylcyclohex-2-en-1-yl)but-3-en-2-one; (E)-4-(2,5,6,6-tetramethylcyclohex-2-en-1-yl)but-3-en-trimethylhexyl acetate; isopropyl 2-methyl butanoate; (E)-3-methyl-4-(2,6,6-trimethylcyclohex-2-en-1-yl)but-3-en-2-one; (3a R,6S,7aS)-3a,4,5,6,7,7a-hexa hydro-1H-4,7-methanoinden-6-yl acetate; (Z)-3-methyl-2-(pent-2-en-1-yl)cyclopent-2-enone; (Z)-3,4,5,6,6-penta methylhept-3-en-2-one; (Z)-1-(1-ethoxyethoxy)hex-3-ene (2E,6Z)-3,7-dimethylnona-2,6-dienenitrile; (Z)-hex-3-en-1-yl methyl carbonate; 3-(4-(tertbutyl)phenyl)-2-methylpropanal; 3,7-dimethylocta-1,6-dien-3-ol; 3,7-dimethylocta-1,6-dien-3-yl acetate; (4E)-9-hydroxy-5,9-dimethyl-4-decenal; 2-methyl-4-oxo-4H-pyran-3-yl isobutyrate; ethyl 2-methylpentanoate; 2,6-dimethylhept-5-enal; 2-isopropyl methylcyclohexanol; 2-isopropyl-5-methylcyclohexanone; 1-((1S,8aS)-1,4,4,6-tetramethyl-2,3,3a,4,5,8-hexahydro-1H-5,8a-methanoazulen-7-yl)ethanone; undecan one; methyl non-2-ynoate; 6,6-dimethoxy-2,5,5-trimethylhex-2-ene; 4-(4-methylpent en-1-yl)cyclohex-3-enecarbaldehyde; 2-(2-(4-methylcyclohex-3-en-1-yl)propyl)cyclopentanone; 2-methyl-6-methyleneoct-7-en-2-yl acetate; (E)-methyl non-2-enoate; (Z)-3,7,11-trimethyldodeca-1,6,10-trien-3-yl acetate; (Z)-3,7-dimethylocta-2,6-dien-1-yl acetate; 6,8-dimethylnonan-2-ol; (Z)-non-6-enal; 3-(4-isobutyl-2-methylphenyl)propanal; 4-(tert-pentyl)cyclohexa none; 2-ethyl-N-methyl-N-(m-tolyl)butanamide; 2-methyl-4-methylene-6-phenyltetrahydro-2H-pyran; 2-cyclohexylidene-2-phenylacetonitrile; 2-cyclohexylidene-2-(o-tolyl)acetonitrile; 2,2-dimethyl-2-pheylethyl propanoate; 1-methyl-4-(4-methylpent-3-en-1-yl)cyclohex-3-enecarbaldehyde; 6-(sec-butyl)quinoline; (E)-2-ethyl-4-(2,2,3-trimethylcyclopent-3-en-1-yl)but-2-en-1-ol; 4-(4-hydroxyphenyl)butan-2-one; 2,2,5-trimethyl-5-pentylcyclopentanone; 2,2,2-trichloro-1-phenylethyl acetate); ROSALVA (dec-9-en-1-ol; (1-methyl-2-(5-methylhex-4-en-2-yl)cyclopropyl)-methanol; 4-methylene-2-phenyltetrahydro-2H-pyran; 2-(1-(3,3-dimethylcyclohexyl)-ethoxy)-2-methylpropyl cyclopropanecarboxylate; 3-(4-isobutylphenyl)-2-methylpropanal; 1-(spiro[4.5]dec-6-en-7-yl)pent-4-en-1-one; (E)-5-methylheptan-3-one oxime; (E)-6-ethyl-3-methyloct-6-en-1-ol; (E)-2-((3,5-dimethylhex-3-en-2-yl)oxy)-2-methylpropyl cyclopropanecarboxylate; 1-methyl-4-propan-2-ylcyclohexa-1,4-diene; 1-methyl-4-(propan-2-ylidene)cyclohex-1-ene; 2-(4-methylcyclohex-3-en-1-yl)propan-2-yl acetate; 3,7-dimethyloctan-3-ol; 2,6-dimethyloctan-2-ol; oxacyclohexadecan-2-one; (E)-tridec-2-enenitrile; (E)-4-methyldec-3-en-5-ol; 2,2,5-trimethyl-5-pentylcyclopentanone; (2,2-dimethoxyethyl)benzene and 2-(2,4-dimethylcyclohexyl)pyridine).

7. The consumer product according to claim 1, wherein the shell comprises a material selected from the group consisting of: aminoplast resins, such as melamine-formaldehyde resins, urea-formaldehyde resins and melamine-urea-formaldehyde resins, polyurea resins, poly(meth)acrylate resins, polyester resins, polysaccharides, proteins, polypeptides, silicon oxides and organosiloxanes.

8. The consumer product according to claim 1, selected from the group consisting of: liquid detergents, hard surface cleaners, shampoos, shower gels, liquid soaps, dish washing liquids and fragranced products comprising ethanol.

9. A method for obtaining a consumer product, in particular a consumer product according to claim 1, the method comprising the steps of:
a) Selecting a transparent or translucent product base;
b) Adding a plurality of microcapsules in the form of a slurry, so that the total surface area of the plurality of microcapsules comprised in one litre of consumer product is from 0.02 to 0.27 m², preferably from 0.02 to 0.12 m²;
c) Dispersing the plurality of the microcapsules.

10. A method of utilizing a plurality of microcapsules in a consumer product, the microcapsules comprising a core and a shell around the core, the core comprising at least one functional material, wherein the mean total surface area of the plurality of microcapsules comprised in one litre of the consumer product is from 0.02 to 0.27 m².
